(19) United States
(12) Patent Application Publication
(10) Pub. No.: US 2011/0305647 A1
(43) Pub. Date: Dec. 15, 2011

(54) USE OF A POLYSACCHARIDE WHICH IS EXCRETED BY THE VIBRIO DIABOLICUS SPECIES FOR THE REGENERATION AND PROTECTION OF THE PERIODONTIUM

(75) Inventors: Karim SENNI, Aulnay-sous-Bois (FR); Corinne SINQUIN, Nantes (FR); Sylvia COLLIEC-JOUAULT, Nantes (FR); Gaston-Jacques GODEAU, Antony (FR); Jean GUEZENNEC, Plouzane (FR)

(73) Assignee: INSTITUT FRANCAIS DE RECHERCHE POUR L'EXPLOITATION DE LA MER (IFREMER), Issy Les Moulineaux (FR)

(21) Appl. No.: 13/162,804

(22) Filed: Jun. 17, 2011

Related U.S. Application Data
(62) Division of application No. 12/096,588, filed on Jun. 6, 2008, now abandoned.

(30) Foreign Application Priority Data
Dec. 7, 2005 (FR) FR 05 12415

(51) Int. Cl. A61K 31/715; A61K 38/18; A61P 29/00; A61P 1/04; A61K 38/39; A61P 1/02

(52) U.S. Cl. 424/49; 514/54; 514/17.2; 514/7.6; 514/8.9; 514/8.2; 514/9.1; 514/8.8

(57) ABSTRACT

The invention relates to the use of a polysaccharide which is excreted by the Vibrio diabolicus species for the regeneration and protection of the non-mineralised connective tissue of the periodontium.

The present invention relates to the regeneration of the non-mineralized connective tissue of the periodontium.

Exopolysaccharide (EPS)-producing bacteria have been isolated from microorganisms originating from deep hydrothermal ecosystems. HE800 is an EPS produced by the Vibrio diabolicus strain. Its weight-average molecular mass is approximately 800 000 g/mol in the native state. It is characterized by an original linear repeating oside sequence consisting of 4 oside residues:

[(-3)-DGlcNacβ(1-4)DGlcAβ(1-4)DGlcAβ(1-4)DGalNacα(1-)]n

HE800 has been described in the International application in the name of IFREMER published under number WO 98/38327 and also in the following articles: Raguénès et al., Int J Syst Bact, 1997, 47, 989-995 and Rougeaux et al., Carbohyd. Res., 1999, 322, 40-45. Many applications have been described for this exopolysaccharide. By way of example of an application, mention may be made of International application WO 02/02051, which describes the beneficial properties of HE800 in bone healing. No application for HE800 is known to date with regard to the regeneration of the non-mineralized connective tissue of the periodontium.

The periodontium is a collection of tissues, the purpose of which is to support and maintain the tooth in its alveolus. It is comprised of two soft tissues, namely the gum and the periodontal ligament (desmodontium), and two calcified tissues, the cement and the alveolar bone. This organ has the particularity of being in an environment where it is subjected to continual attacks (bacterial, mechanical, chemical). The oral cavity is in fact a moist medium harboring a commensal bacterial flora. The integrity of the periodontium depends essentially on the equilibrium between the oral tissues and this bacterial flora. Any destabilization of this relationship promotes the proliferation of a pathogenic flora that can lead to the destruction of the periodontal tissues. In order to meet these constraints, the periodontium is constantly undergoing remodeling.

The gum is the tissue that covers the periodontium, it thus constitutes a protection, against bacterial attacks, for the periodontal elements that it covers (cement, desmodontium, and alveolar bone). Histologically, this tissue is composed of a connective tissue covered by an epithelium of ectodermal origin. The gingival connective tissue is composed of a gingival extracellular matrix which is very similar to that of the dermis in terms of the macromolecular content. Unlike the dermis, the gum has a direct relationship with various mineralized tissues and has several types of collagen fibers that link the gum to the alveolar bone, to the cement and to other fibers linked to the neighboring tooth.

Fibrillar collagens represent 50% to 60% of the proteins found in the gingival connective tissue. Phenotypic analyses show that these collagens are made up of 91% type I, 8% type III and less than 1% type V.

The extracellular matrix constitutes the framework of connective tissue. It gives the tissue its shape, its mechanical strength and its flexibility and performs important physiological functions. It is also necessary for maintaining the differentiated state of the cells which synthesize and remodel it. Through membrane receptors such as integrins, it is in close association with resident cells such as fibroblasts, which makes it possible, depending on its state, to control the migration, proliferation or metabolic activities thereof. In return, these cells can remodel the matrix that surrounds them. They can then express proteases in order to degrade it or to resynthesis new matrix components. The extracellular matrix is therefore in constant equilibrium (degradation-resynthesis). This equilibrium can be irreparably disturbed during certain pathological conditions. In fact, in inflammatory syndromes, the destructive capacity of the resident cells is exacerbated under the influence of the inflammatory cells, the latter, after activation, also degrading the matrix. Other pathological conditions may contribute to disturbing the matrix dynamics, such as fibroses during which the expression of one or more matrix components is exacerbated.

In this context, correct restructuring of the extracellular matrix is responsible for tissue regeneration. The organization of the collagen network is an essential element of this tissue restructuring. This is because these collagens constituent the predominant protein class of extracellular matrices, and in particular those of the periodontium.

Unlike the dermis, the gum is subjected to considerable and constant remodeling. This remodeling is the consequence of the coexistence between the bacterial plaque that is deposited on the tooth and the gingival tissue, and mechanical stress to which the gum is subjected during chewing. These factors more or less directly affect the gingival fibroblasts which represent the majority of the cells present in the gingival connective tissue. These fibroblasts are to a large extent responsible for the remodeling observed in the healthy gum. In order to maintain the attachment of the gum to the cement and the alveolar bone, the gingival fibroblasts should be capable of constantly responding to the stresses that are exerted on the tissue that harbors them.

This constant activation is reflected by the gingival fibroblasts having a very high phenotypic heterogeneity. A distinction can in particular be made between myofibroblasts and fibroblasts; myofibroblasts proliferate in the case of an inflammatory process. The fibroblast is the key cell in tissue homeostasis.

While this fibroblast heterogeneity is an advantage under physiological conditions, it can prove to be extremely detrimental in the case of periodontal pathological conditions that set in over the long term. In fact, any long-lasting impairment of cellular equilibrium can lead to the unwanted activation of certain cell phenotypes, for instance the proliferation of myofibroblasts at the expense of fibroblasts.

High-molecular-weight hyaluronic acid is commonly used to treat many oral pathological conditions.

EP0444492 describes the use of hyaluronic acid for treating inflammatory diseases of the oral cavity, such as gingivitis. WO2005000321 describes the use of hyaluronic acid for treating oral cavity aphthas. Hyaluronic acid is used in various formulations for these treatments; by way of example, mention may be made of Gengigel® which is in the form of a spray or mouthwash.

Hyaluronic acid, which is a product of animal origin, is produced from an animal extract or by genetic engineering.

The object of the present invention is to identify a compound capable of preserving periodontal tissue homeostasis and/or of promoting the restructuring, i.e. the restoring, of an altered collagen network of non-mineralized connective tissues of the periodontium, and of promoting gingival fibroblast proliferation in order to reestablish the gingival homeostasis.

Such a composition will thus have a protective and regenerative activity on non-mineralized connective tissue of the periodontium and will facilitate the reestablishment of tissue homeostasis.

The inventors have demonstrated, surprisingly and unexpectedly, that a polysaccharide having a weight-average molar mass of between 500 000 and 2 000 000 g/mol, characterized by a linear repeating oside sequence comprising the 4 oside residues:

[(-3)-DGlcNacβ(1-4)DGlcAβ(1-4)DGlcAβ(1-4)DGalNacα(1-)]

has the following properties: it induces fibroblast strain selection, it stimulates fibroblast mobilization and proliferation in the extracellular matrix, it accelerates collagen fibrillation and thus promotes reconstruction of the connective matrix. This means that this polysaccharide accelerates the regeneration by accelerating the restructuring of the connective tissue. It makes it possible to achieve complete regeneration such that the appearance of pathological situations of fibrotic or inflammatory type is prevented.

This polysaccharide makes it possible to reconstruct the collagen network of non-mineralized connective tissue of the periodontium, and it constitutes a support allowing the adhesion and cell proliferation of gingival fibroblasts.

Thus, by virtue of its properties, the polysaccharide is particularly suitable for the following applications: the regeneration of the connective tissue of the periodontium as well as the treatment of oral pathological conditions, in particular those linked to an inflammatory state or to a traumatic state.

In addition, by virtue of these same properties, the polysaccharide enables the production of collagen matrix with improved properties. In fact, the collagen network of collagen matrices comprising the polysaccharide exhibits better resistance against physical factors such as temperature and mechanical stresses. Finally, it promotes the culture of gingival fibroblasts, and allows the preparation of gum substitutes.

A subject of the present invention is the use of a polysaccharide or of a salt of this polysaccharide having a weight-average molar mass of between 500 000 and 2 000 000 g/mol, preferably between 700 000 and 900 000 g/mol, characterized by a linear repeating oside sequence comprising the following 4 oside residues:

[(-3)-DGlcNacβ(1-4)DGlcAβ(1-4)DGlcAβ(1-4)DGalNacα(1-)]

for the production of a composition, of a medicament or of a medical device having a protective and/or regenerative activity on the non-mineralized connective tissue of the periodontium.

Typically, the polysaccharide may be in the form in of a salt.

Typically, the polysaccharide is a polysaccharide excreted by the Vibrio diabolicus species or a derivative obtained therefrom. Methods of preparation have been described in the following documents: WO 98/38327, Raguénès et al., Int J Syst Bact, 1997, 47, 989-995 and Rougeaux et al., Carbohyd. Res, 1999, 322, 40-45. By way of example, derivatives having a weight-average molar mass of between 500 000 and 2 000 000 g/mol can be obtained by partial depolymerization, by bridging and/or by chemical modifications, in particular by sulfatation and/or by acetylation. By way of example, WO0046252 describes a method for bridging hyaluronic acid; typically, this method may be adapted so as to generate bridged derivatives of the polysaccharide excreted by the species Vibrio diabolicus.

One embodiment of the invention relates to the production of a composition, of a medicament or of a medical device for treating an oral pathological condition of the non-mineralized connective tissue of the periodontium.

In particular, the oral pathological condition is linked to an inflammatory state or to a traumatic state.

Preferably, the oral pathological condition is chosen from the group consisting of periodontitis, gingivitis, gingival fibrosis, gingival recession, aphtha, recurrent oral aphthosis, aphthous diseases, and bullous pathological conditions.

Typically, the composition or the medicament produced is for topical administration at the periodontal level.

The term “topical composition” is intended to mean a composition which acts at a given point and which can be directly applied to the oral mucosal.

The composition and the medicament may be in the form of a topical composition for oral use, in particular in the form of a gel, a solution, an emulsion or a spray. Typically, a topical composition according to the invention is produced in a manner known per se. By way of example, a topical composition according to the invention contains the polysaccharide at a concentration of between 0.005% and 10% by weight relative to the total weight of the composition, more preferably at a concentration of between 0.01% and 5% by weight.

A gel according to the invention may comprise sorbitol, maltitol, xylitol and/or sodium carboxymethylcellulose.

This same composition or this same medicament may be used to impregnate an oral dressing.

One embodiment of the invention relates to a toothpaste, a mouthwash, a spray, a denture adhesive and an oral dressing comprising the polysaccharide. Typically, those skilled in the art may use the customary techniques for developing these products. By way of example, a toothpaste, a mouthwash or a spray according to the invention contains the polysaccharide at a concentration of between 0.005% and 1% by weight relative to the total weight of the composition, more preferably at a concentration of between 0.01% and 0.1% by weight. A toothpaste according to the invention may comprise, by way of example, one or more of the following compounds: sorbitol, maltitol, xylitol and/or sodium carboxymethylcellulose. Typically, a mouthwash according to the invention may contain one or more of the following excipients: polysorbate 60, sodium saccharine, methyl salicylate, essence of clove; essence of anis, essence of eucalyptus, citric acid, menthol. A mouthwash according to the invention may also contain an additional active agent such as, for example, hexatidine.

For all the compositions, medicaments, toothpastes, mouthwashes, sprays and denture adhesives according to the invention, the polysaccharide may be used as the only active agent, or accompanied by other active agents such as, for example, an antibacterial agent, an antibiotic, vitamins or trace elements.

According to another embodiment, the invention relates to a collagen matrix comprising the polysaccharide according to the invention.

Typically, the collagen of the matrix is a collagen chosen from the group consisting of collagen type I, III and V or of a mixture thereof. Preferably, the collagen is a collagen type I.

Typically, in order to produce such a collagen matrix, those skilled in the art will use the techniques commonly used for the production of collagen matrices from acid-soluble fibrillar collagens. In the presence of the polysaccharide according to the invention, acid-soluble fibrillar collagens naturally form fibrils after neutralization of the pH. Alternatively, the collagen matrix according to the invention may be obtained by bridging of the polysaccharide according to the invention with the collagen. In order to carry out the bridging, those skilled in the art will use the techniques commonly used for bridging polysaccharides with collagen. EP1374857 is an illustration of a bridging technique which can be used.

Advantageously, the matrix may also comprise a growth factor which promotes colonization of the matrix by the gingival fibroblasts and the reconstruction of the connective tissue.

Preferably, the growth factor may be chosen from the group consisting of TGF-beta, PDGF, FGFs, BMPs (bone morphogenetic proteins), VEGF and CTGF (connective tissue growth factor).

Typically, the matrix may serve as a resorbable medical device or as an implant. Such a matrix will allow the mechanical and functional replacement of damaged structures with a minimum of adverse reactions. Once implanted into the tissue to be regenerated, this matrix will serve as a guiding structure and will pave the way for the regenerative potential of the tissue. The matrix will promote ordered penetration of the fibroblasts after grafting of the matrix, while at the same time prompting these same fibroblasts to produce their own extracellular matrix.

According to a preferred embodiment of the invention, the matrix may comprise gingival fibroblasts so as to constitute a gum substitute. This substitute may be implanted in vivo. Advantageously, it is the gingival fibroblasts of the patient on whom the graft has to be carried out which serve to colonize the matrix.

According to another embodiment, the invention relates to a cell culture support, characterized in that the surface of the support on which the cells are cultured comprises the polysaccharide according to the invention.

Typically, the polysaccharide is in the form of a film, a membrane or a three-dimensional honeycombed structure.

According to another embodiment, the invention relates to a method of culturing gingival fibroblasts, characterized in that said fibroblasts are cultured on a matrix according to the invention or on a support as described above.

The content of all the documents cited should be considered to be part of the present description.

The present invention will be illustrated more clearly hereinafter by means of the examples which follow. These examples are given only by way of illustration of the subject of the invention, of which they no way constitute a limitation.

### EXAMPLES

### 1 Materials and Methods

1.1. Preparation of the Exopolysaccharide HE800 from Cultures of Vibrio diabolicus (HE800 Strain)

Methods for preparing HE800 have been described in the following documents: WO 98/38327, Raguénès et al., Int J Syst Bact, 1997, 47, 989-995 and Rougeaux et al., Carbohyd. Res, 1999, 322, 40-45.

a) Cultures of Vibrio diabolicus

The HE800 strain is cultured on 2216E medium [Oppenheimer, J. Mar. Res. 11, 10-18, (1952)] enriched with glucose (30 g/l). The production is carried out at 30° C. and at pH 7.4 in a 2-liter fermenter containing 1 liter of the 2216E-glucose medium. After culturing for 48 hours, the must has a low viscosity (of the order of 40 centipoises at 60 rpm).

b) Purification of the Exopolysaccharide

The bacteria are separated from the must by centrifugation at 20 000 g for 2 hours, and the polysaccharide is then precipitated from the supernatant with pure ethanol, and several ethanol/water washes are then carried out with increasing proportions of ethanol, according to the method described by Talmont et al. [Food Hydrocolloids 5, 171-172 (1991)] or Vincent et al. [Appl. Environ. Microbiol., 60, 4134-4141 (1994)]. The polysaccharide obtained is dried at 30° C. and stored at ambient temperature. 2.5 g of purified polysaccharide per liter of culture were thus obtained.

1.2. Obtaining Fibroblasts

The experiments were carried out on fibroblasts of dermal origin and fibroblasts of gingival origin. These two types of fibroblasts adopt a very similar behavior with respect to HE800; consequently, the results obtained with the dermal fibroblasts can be extrapolated to the gingival fibroblasts.

1.2.1) Culture Media:

The cultures are carried out in a “complete” medium composed of Dulbecco MEM Glutamax I containing 100 U/ml of penicillin, 100 μg/ml of streptomycin and 2 μg/ml of fungizone (Gibco BRL Cergy Pontoise, France) supplemented or not supplemented (deficient medium) with fetal calf serum (FCS).

1.2.2) Origin of the Tissue Samples:

The dermal biopsies used are placed in culture within 3 hours of them being taken by the practitioner. The samples used are obtained after circumcision, from foreskins of clinically normal children. The gingival biopsies are taken from young patients (under 30 years old) with no pathological conditions. The biopsies are taken from gum attached to premolars extracted for orthodontic reasons. In addition, these gums are declared clinically normal by the practitioner. These biopsies are tissue remnants detached during the extraction and which have required no modification of the intervention.

1.2.3) Culturing:

The dermal and gingival samples are rinsed twice in a DMEM medium containing a higher than normal concentration of antibiotics (6× penicillin, 4× streptomycin, 2× fungizone) and then they are cut up into very small explants (≈2 mm²). These explants are placed, using a sterile Pasteur pipette or with the tip of a scalpel, in a 25 cm² culture flask, with the parenchymal side on the plastic. The dish is then stood up and left in this position for 15 minutes so that the explants adhere, dry, to the plastic.

The explants that have adhered are covered with a few drops of DMEM supplemented with 20% fetal calf serum (FCS). The culture dish is then placed in an incubator at 37° C. overnight, in an atmosphere composed of 5% CO₂ and 95% air. The following day, the supernatant is replaced with fresh medium containing 20% FCS; it is subsequently renewed every week. After three weeks, the fibroblasts have completely colonized the bottom of the dish (the keratinocytes present in the explant do not adhere under these culture conditions); subculturing is then carried out. The explants are removed using forceps, and the cells are rinsed twice with PBS and then trypsinized (trypsin-EDTA, Gibco). The trypsinization is then stopped by adding DMEM containing 10% FCS. The cells are counted on a counter (Coulter) and then reseeded into several culture dishes. They are, at this time, considered to be first passage and are maintained in a complete medium containing 10% FCS. When the cells are again confluent, another passage is carried out according to the same procedure, and this is continued up to the start of the experiments.

### 1.3. Preparation of Films and Culturing of Fibroblasts

1.3.1) Preparation of HE800 Films and Culturing of Fibroblasts

Surfacting is carried out by depositing 200 μl of a 2 mg/ml solution of HE800 at the bottom of the culture wells (24-well dish, 2 cm²). The culture dish is placed under a culture hood on a hotplate set to 37° C., for at least 5 hours. After evaporation, an HE800 film forms at the bottom of the dish. The gingival fibroblasts are seeded at a rate of 10 000 cells per well and cultured for 7 days. The cells are counted each day, some wells are fixed for the morphological study and the immunodetection of smooth muscle α-actin.

1.3.2) Preparation of Collagen Films and of Collagen-HE800 Composite Films and Culturing of Fibroblasts:

The collagen used is an acid-soluble collagen type I (2 mg/ml) obtained from rat tail (Institut Jacques Boy, Reims). Surfacting of the culture dishes is carried out by depositing 200 μl of a mixture of collagen (40 μg in total) and HE800 (5, 50 or 200 μg in total).

The culture dish (24-well dish, or labtek, 2 cm² per well) is placed under a culture hood on a hotplate set to 37° C., for at least 5 hours. After evaporation, a film of collagen with or without HE800 forms at the bottom of the dish. Fibroblasts are seeded onto these films in order to be sure of the biocompatibility of the new culture surface.

1.3.3) Characterization of the Structure of the Films:

The collagen films and the composite films are fixed with absolute ethanol at −20° C. and then rehydrated so as to be stained with Sirius red (Junquera staining, collagen-specific). Thus, in Sirius red, all collagens are stained under transmitted light, but only correctly fibrillated collagens are capable of deviating polarized light.

1.4. Culturing in Lattices (Collagen Matrix): Preparation of Equivalent Non-Mineralized Connective Tissue

The lattices are made up with the same collagen I as that used to form the collagen films. After neutralization of the acid solution of collagen (3 mg/lattice), the gel containing the cells, and which is undergoing polymerization, is poured into a Petri dish 5 cm in diameter. HE800 is added to the collagen before the addition of the cells, at a rate of 150 μg, 300 μg or 600 μg per lattice (respectively 5%, 10% and 20% of the total amount of the collagen).

1.4.1) Preparation of the Stock Solution:

| Components | Amount | |
|---|---|---|
| DMEM (powder) | 5 | g |
| NaHCO₃ | 1.1 | g |
| Nonessential AA (100×) | 5 | ml |
| BiΔ H₂O (sterile) | 17 | ml |

Filter

| | | |
|---|---|---|
| BiΔ H₂O (sterile) | 250 | ml |

10 ml of fetal calf serum are added per 50 ml of stock solution.

1.4.2) Preparation of the Lattices:

All the steps for preparing the lattice are carried out in ice.

| | | |
|---|---|---|
| Stock solution (FCS) | 2.75 | ml |
| Collagen (2 mg/ml) | 1.5 | ml |
| NaOH (0.1N) | 0.25 | ml |
| Cells (300 000/ml) | 0.5 | ml |

The lattice is shaken then poured into the Petri dish and then left for 5 min at 37° C.
After 1 h, the dishes are slightly shaken in order to detach the lattices from the edges.
The culture media are changed every week.

1.4.3) Characterization of the Lattices (Collagen Matrices):

At various culture times (11 and 40 days), the lattices are recovered, fixed in paraformaldehyde, and then prepared for paraffin embedding. Sections 7 μm thick are then cut on a microtome. Specific staining of these sections makes it possible to observe and study the structure and the cellularity of the reconstructed connective tissue. Some of the parameters demonstrated can subsequently be studied by image analysis and thus be quantified. The quality of the collagen fibrillation is observed after staining with Sirius red; the cellularity of the equivalent connective tissue could be estimated by an image analysis after staining the sections with hemalun-eosin.

1.4.4) Determination of the Number of Fibroblasts Contained in the Lattices:

Hemalun-eosin staining makes it possible to distinguish the cells from the matrix which surrounds them. This is because hemalun stains the cell nuclei blue-black, whereas eosin stains the cytoplasms and the extracellular structures (eosinophilic) more or less intensely red. The contrast thus created makes it possible to distinguish each cell under a microscope equipped with a CDD camera connected to a semi-automatic image analyzer. The cells which are in the fields defined by the microscope magnification are then counted in the lattices at 11 and 40 days. About ten fields per section were analyzed. Two groups of cells can thus be differentiated according to their geographical situation: firstly, the cells which are inside the lattice (collagen matrix) and, secondly, the cells which are at the periphery of the lattice. In order to calculate the cellularity of the equivalent connective tissue, each lattice is considered to be cylindrical, the periphery of the lattice being defined as a crown 10 μm thick (equivalent to the diameter of two cell strata) representing 2% of the total volume of the lattice.

1.5. Indirect Immunodetection of Smooth Muscle α-actin

The fixed cells are repermeabilized in 70% ethanol (20 min) and then rehydrated in PBS (10 min). The endogenous peroxidases are blocked with a methanol (30%), H₂O₂ (0.3%) solution. This operation is followed by rinsing with PBS (2 min), and then by blocking of the nonspecific antigenic sites with a PBS/1% skimmed milk solution (1 h). The cultures are then incubated with a primary antibody (mouse IgG) directed against human α-actin (1/30; 50 min) and then rinsed with PBS (3×10 min). The cells are then incubated in the dark for 60 min with a biotinylated anti-mouse IgG antibody (1/200), rinsed with PBS (3×10 min), and then incubated with peroxidase-coupled streptavidin (1/200).

After rinsing (PBS 3×10 min), the peroxidase activity is revealed with 3,3′-diaminobenzidine in a Tris/HCl buffer (100 mM, pH 7.2-7.4) containing 0.1% of H₂O₂ (15 min, in the dark). The peroxidase activity causes a brown fibrillar material to appear (corresponding to the α-actin microfilaments) in the cytoplasm of the positive cells.

The products used come from the company Dako. The controlled experiments concerning the immunodetection of smooth muscle α-actin were carried out by omitting the primary antibody and/or by using a secondary antibody of an animal species other than that which made it possible to obtain the primary antibody.

### 2. Results and Discussion

2.1. Proliferation of Gingival Fibroblasts Cultured on HE800 Film

The culture surfaces were treated with HE800 in order to form a polysaccharide film at the bottom of the dishes. During the first days of culture (days 2 and 4), it is observed that the number of cells seeded in the HE800-coated dishes is much lower than the number of those in the control dishes (Table I). On the other hand, on the last day of the experiment, an inversion of these results is noted (Tables I and II). The curves presented show that the cells cultured on HE800 film observe a lag phase, before entering into the exponential growth phase, which is longer than that expressed by the cells cultured on plastic. Furthermore, although the number of cells in the control cultures reach a plateau at the latest days of the experiment (cf. Table III), the cultures on HE800 film continue to proliferate. The observations made during culturing or after fixing of the cells make it possible to put forward hypotheses as to the cell behaviors expressed under the various culture conditions:

The cultures on HE800 film are characterized, in the first days of culture, by the presence of numerous cells which do not adhere to the support. This nonadhesion may explain the delay in proliferation observed in the cell counts in these cultures.

The control cells are distributed uniformly in the dish, without any particular orientation, whereas the cells seeded on HE800 film become organized in strings at the center of the dish. These results show the effect of HE800 on the cell adhesion. In fact, the cell groupings which are normally observed, in gingival cultures, have no specific orientation. After the first 2 days of culture, these strings of cells begin to form a circular central structure, becoming denser exclusively toward the center (centripedal proliferation). Many cells can also be observed at the periphery of the dish, but with no particular orientation. Some cells may be present in the areas separating the cell groupings, they are isolated and appear to be much more drawn out in length than the other cells of the HE800 or even control dishes.

The immunocytochemical labeling regarding the smooth muscle α-actin shows:

in the controls, many positive cells are next to cells not expressing these microfilaments.

in the cultures in the presence of HE800, the cells present in the central circular formations do not express smooth muscle α-actin; on the other hand, cells expressing this actin isoform can be found at the periphery of the dish.

These results reflect a selection of fibroblast strains; in fact, some cells may not naturally express the membrane receptors required for them to adhere to the HE800 film. Among the nonadherent fibroblast subpopulations are those which express smooth muscle α-actin, i.e. myofibroblasts. In the control experiments (emission of the primary antibody or use of an inappropriate secondary antibody), no positive was observed.

TABLE I
Variation in the number of cells per well during the culture

| | Day 2 | Day 4 | Day 7 |
|---|---|---|---|
| Control | 14 812 | 45 610 | 52 980 |
| HE800 | 10 035 | 36 995 | 64 247 |

TABLE II
Proliferation percentages

| | Day 2 | Day 4 | Day 7 |
|---|---|---|---|
| Control | 100 | 100 | 100 |
| HE800 | 67.75 | 81.11 | 127.27 |

TABLE III
Doubling time (hours)

| | Dt 0-2 | Dt 2-4 | Dt 4-7 |
|---|---|---|---|
| Control | 84.68 | 29.58 | 333.2 |
| [symbol] HE800 | 9522.64 | 25.50 | 90 |

[symbol]

indicates data missing or illegible when filed

### 2.2. Structuring of Collagen Type I in the Presence of HE800

2.2.1) First Observations

In order to prepare the films comprising both collagen and exopolysaccharide HE800, solutions of HE800 and collagen I are premixed before deposition onto the culture dishes. Surprisingly, it was noted that the addition of the bacterial exopolysaccharide to the collagen solution caused the appearance of a dense, white-colored agglomerate. This agglomerate could be spread on a histological slide and then stained with Sirius red, a collagen-specific dye. These histological slides show that the material spread on the slide is effectively stained with Sirius red. Furthermore, the observation of a material causing polarized light to deviate in various directions clearly shows the presence of collagen in its fibrillar form.

2.2.2) Organization of Collagen/HE800 Composite Films:

The various films deposited are composed of:
(1) collagen (40 μg)
(2) collagen (40 μg)+HE800 (50 μg)
(3) collagen (40 μg)+HE800 (200 μg).

Observation of the bottom of the dishes under a microscope shows, for the films (3), the appearance of a dense network composed of long filaments. The films (2) comprise some much shorter fibers, whereas the films (1) comprise virtually none. The 3 films stain with Sirius red, but only the film (3) shows a fibrillar network which causes polarized light to deviate.

These results indicate that HE800 promotes the formation of collagen fibers, but also allows better resistance of the collagen network against physical factors such as temperature and mechanical stresses.

2.3. Non-Mineralized Connective Tissue: Photon and Electron Microscopy

The cells are cultured in a collagen matrix (three-dimensional culture model) in order to mimic as closely as possible the cell/matrix interactions observed in connective tissue. These lattices or equivalent connective tissues are composed of collagen I alone (controls) or of collagen I and HE800 in various proportions (amount of EPS=20, 10 and 5% relative to the amount of collagen contained in the lattice, i.e. 300, 150 and 75 μg, respectively).

2.3.1) Retraction of the Lattices:

The first parameter studied is the rate of retraction of the lattices: the retraction curves for the control lattices and for the lattices comprising HE800 are similar. Despite these similarities, it is noted that the HE800 lattices have a slower retraction rate than the control lattices during the early days of culture. After the 11^{th} day, the retraction of the lattices is almost complete.

2.3.2) Number of Fibroblasts Contained in the Lattices:

The number of cells present in each lattice varies, at the two culture times, between 180 000 and 250 000 cells. The number of cells at the periphery represents 2 to 12% of the total number of cells. These data are compatible with what has been described in the literature for this culture model. The results in Tables IV, V and VI show the number of cells per unit of volume (mm³) present in the entire lattice and in its various regions.

The total volumetric cell densities of the lattices after 11 and 40 days are between 3200 and 5900 cells/mm³ (cf. Table IV). These values are comparable to those found in a normal human connective tissue, as has been previously described (Miller et al., Exp Dermatol. 2003 August; 12(4): 403-11). The physiological cellularity of the control lattices and of the lattices comprising HE800 therefore attests to the validity of the culture model used and to the compatibility of HE800 with this physiological model.

The total cell density (cf. Table IV) of the control lattices does not vary whatever the culture time. At the 11^{th} day of culture, the total cell density of the HE800 lattices is 25 to 40% lower than those of the control lattices. At the 40^{th} day of culture, the cell densities of the control lattices and of the HE800 lattices are equivalent. The variations in the cell densities observed inside the lattices (cf. Table V) reproduce exactly those of the entire lattice. The topological organization of the cells of the peripheral crown (Table VI) on the other hand diverge completely from those of Tables IV and V:
at 11 days of culture, the cell densities of the peripheral crown are 4 times higher than those of the interior of the lattice and show only slight variation between the control equivalent connective tissues and the equivalent connective tissues comprising HE800 (Table VI).
at 40 days of culture, a large decrease in the peripheral cellularity is observed. While this decrease is only 40% for the control lattices, it reaches 100 to 250% for the lattices containing HE800. The cell density of the control lattices remains 3 times higher at the periphery than at the interior (Table IV and VI); on the other hand, these densities are comparable for the HE800 lattices.

The overall cellularity of the HE800 lattices is lower than that of the control lattices early on in the culture, and then becomes equivalent later on in the culture. These variations, which have an effect on the cell densities of the internal regions, can be explained by a stimulation of cell proliferation, or massive migration of peripheral cells to the interior.

In fact, at the periphery of the lattices, the number of cells decreases over the culture time; this decrease is particularly accentuated in the lattices comprising HE800 (decrease by 2 to 3.5 times of the number of cells). This decrease can be explained by a loss of adhesion of the peripheral cells, which detach from the extracellular matrix, and/or a massive migration of these cells to the interior. This explains the overall gains in cellularity, over time, in the lattices containing HE800.

TABLE IV
Cell density in the entire lattice: number of cells per mm³ (between
parentheses: lattice diameter in mm)

| | | | | | Variation between |
|---|---|---|---|---|---|
| Culture time | 11 days | | 40 days | | 11 and 40 days |
| Controls | 5924 | (8.3) | 5695 | (8) | −4% |
| HE800 20% | 4468 | (8) | 6150 | (8) | +27% |
| HE800 10% | 3899 | (9.7) | 6000 | (8) | +35% |
| HE800 5% | 3491 | (1.03) | 5023 | (8) | +30% |

TABLE V
Cell density inside the lattice: number of cells per mm³

| | | | Variation between |
|---|---|---|---|
| Culture time | 11 days | 40 days | 11 and 40 days |
| Controls | 5568 | 5520 | −3% |
| HE800 20% | 4153 | 6085 | +22% |
| HE800 10% | 3517 | 5938 | +41% |
| HE800 5% | 3220 | 5005 | +36% |

TABLE VI
Cell density at the periphery of the lattice: number of cells per mm³

| | | | Variation between |
|---|---|---|---|
| Culture time | 11 days | 40 days | 11 and 40 days |
| Controls | 20 134 | 14 382 | −40% |
| HE800 20% | 23 367 | 6531 | −258% |
| HE800 10% | 22 161 | 9979 | −122% |
| HE800 5% | 17 731 | 5939 | −199% |

Conclusion: HE800 promotes the proliferation of dermal fibroblasts in the extracellular matrix and/or promotes their mobilization, i.e. the selection, migration and massive penetration of the peripheral cells.

### 2.4.3) State of the Collagen Matrix:

2.4.3.1) Photon Microscopy

Sirius-red staining (Junquera staining) makes possible to specifically stain collagens; in the skin, for example, its collagens appear in the form of a red-colored, loose filamentous structure.

The Sirius-red stainings of the histological sections after observation under transmitted light and polarized light show that the addition of HE800 during the formation of the lattice allows the formation of a matrix which is much more dense and after much shorter periods of time than in the control lattices.

For example, the density of the control collagen matrix after 40 days of culture is equivalent to that observed in the collagen matrices formed in the presence of HE800 at 11 days of culture. This effect on the density is much greater at the lowest doses (10%, 5%).

2.4.3.2) Electron Microscopy

Electron microscopy was carried out on equivalent connective tissues cultured for 11 days. The cells were seen to have a good ultrastructural state, whether in the controls or in the lattices formed in the presence of the various concentrations of HE800.

No collagen fibers could be observed in the control lattices; the lattices comprising 20% of HE800 make it possible to observe some fibrillar elements held in a gel consisting of the exopolysaccharide. The lattices comprising 10% and 5% of exopolysaccharides are very different; specifically, numerous collagen fibers are present, they are distributed throughout the lattice and some exhibit a periodic striation. These collagen fibers or fibrils are trapped in the gel consisting of the HE800.

Conclusion: The HE800 accelerates collagen fibrillation and promotes the constitution of an extracellular matrix.

1.-17. (canceled)

18. A method for treating non-mineralized connective tissue of the periodontium comprising a step of: contacting the periodontium with a polysaccharide or a salt thereof having a weight-average molecular weight of 500,000 to 2,000,000 g/mol, wherein the polysaccharide or salt thereof has a linear repeating osidic sequence:
[(-3)-DGlcNacβ(1-4)DGlcAβ(1-4)DGlcAβ(1-4)DGlcNacα(1-)].

19. The method according to claim 18, wherein said polysaccharide is a polysaccharide excreted by a bacterium of the Vibrio diabolicus strain or a derivative of this polysaccharide.

20. The method according to claim 18, wherein the method comprises treating a subject suffering from an oral pathological condition of non-mineralized connective tissue of the periodontium.

21. The method according to claim 20, wherein the oral pathological condition is associated with an inflammatory state or a traumatic state.

22. The method according to claim 20, wherein the oral pathological condition is selected from the group consisting of periodontitis, gingivitis, gingival fibrosis, gingival recession, aphtha, recurrent oral aphthosis, aphthous diseases, and bullous diseases.

23. The method according to claim 18, wherein the polysaccharide or salt thereof is administered topically at the periodontal level.

24. The method according to claim 18, wherein the polysaccharide or salt thereof is formulated in the form of a gel, a solution, an emulsion or a spray.

25. The method according to claim 18, wherein the polysaccharide or salt thereof is formulated in the form of a toothpaste, a mouthwash, a mouthspray or a denture adhesive.

26. The method according to claim 18, wherein the polysaccharide or salt thereof is in a medical device.

27. The method according to claim 26, wherein the medical device is an oral dressing, an oral resorbable device or an oral implant.

28. The method according to claim 26, wherein the medical device is a gum substitute.

29. The method according to claim 26, wherein the medical device comprises a collagen matrix.

30. The method according to claim 29, wherein the collagen matrix comprises a collagen selected from the group consisting of collagen type I, collagen type III, collagen type V and mixtures thereof.

31. The method according to claim 29, wherein the collagen matrix comprises collagen type I.

32. The method according to claim 29, wherein the collagen matrix further comprises at least one growth factor that promotes colonization of the matrix by gingival fibroblasts and reconstruction of the non-mineralized connective tissue of the periodontium.

33. The method according to claim 32, wherein the at least one growth factor is selected from the group consisting of transforming growth factor beta (TGF-beta), platelet-derived growth factor (PDGF), fibroblast growth factor (FGF), vascular endothelial growth factor (VEGF), bone morphogenetic proteins (BMPs), and connective tissue growth factor (CTGF).

34. The method according to claim 32, wherein the collagen matrix further comprises gingival fibroblasts.
